(19) United States
(12) Patent Application Publication
(10) Pub. No.: US 2009/0088717 A1
(43) Pub. Date: Apr. 2, 2009

(54) ABSORBENT NURSING PAD ADHESIVE COVER

(76) Inventors: Charles J. Pawloski, Cary, NC (US); Tina M. Bruce, Cary, NC (US)

Correspondence Address: GREENBERG TRAURIG, LLP, MET LIFE BUILDING, 200 PARK AVENUE, NEW YORK, NY, 10166, US

(21) Appl. No.: 12/240,339

(22) Filed: Sep. 29, 2008

Related U.S. Application Data
(60) Provisional application No. 60/975,996, filed on Sep. 28, 2007.

(51) Int. Cl. A61F 13/15

(52) U.S. Cl. 604/385.07; 604/385.01

(57) ABSTRACT

A reusable, washable selectively adhesive cover for use by lactating women in combination with an absorbent nursing pad, said cover comprising a plurality of laminated silicone compound membranes, an outer laminate of a generally higher durameter than an inner laminate.

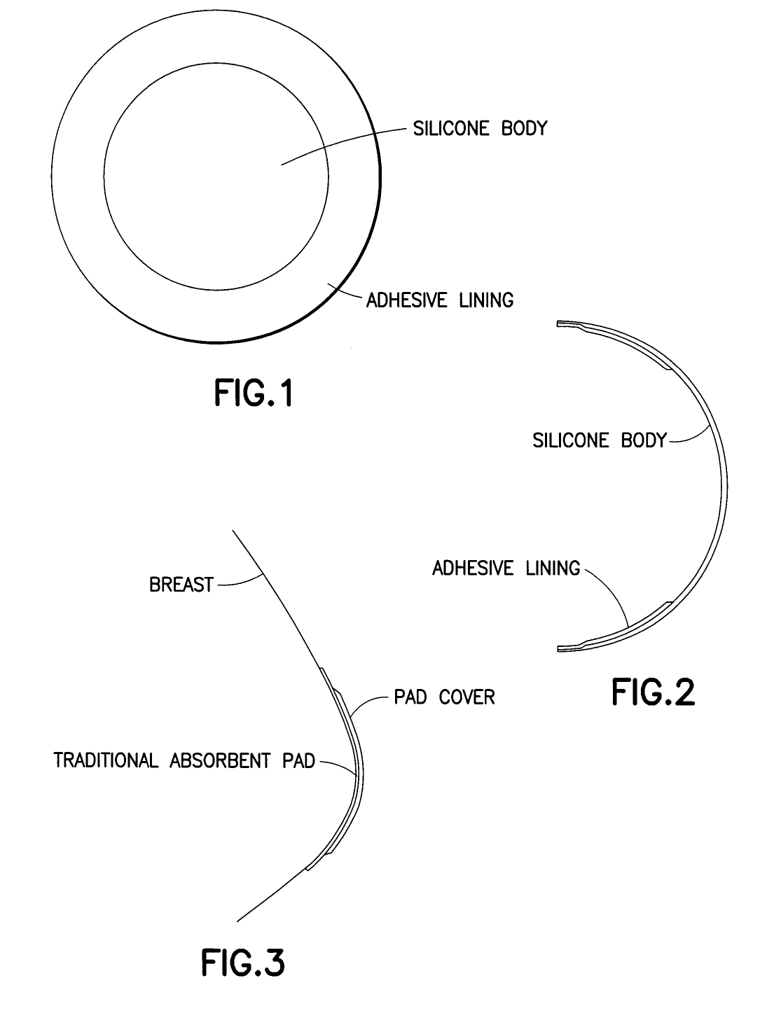

### BACKGROUND OF THE INVENTION

A reusable, washable selectively adhesive cover for use by lactating women in combination with an absorbent nursing pad. The cover comprises a plurality of laminated silicone compound membranes; the outer laminate of a generally higher durameter than the inner laminate. The cover is impermeable to liquid but permeable to gases. The surface of the cover placed against the breast of the user (the inner laminate) comprises an inherently adhesive outer circumference and non-adhesive interior. The cover is envisioned to be adhered to the breast of the user over a traditional absorbent nursing pad of any standard size.

The cover may be held in place with an article of clothing, but is sufficiently adhesive that clothing is not required for use. The cover is thin and translucent and is relatively unnoticeable under clothing as its outer surface is smooth.

As used herein, “reusable” means possible to use again.

“Washable” means capable of being washed without significant change to structure and function.

“Selectively adhesive” means capable of adhering in certain regions.

“Laminated” means bonded.

Suitable “silicone compound membranes” include, but are not limited to GE Silicones 60-10 material and Wacker Silgel material.

“Impermeable to liquid” means substantially not capable of allowing liquid to pass through.

“Permeable to gases” means substantially capable of allowing gases to pass through.

“Inherently adhesive outer circumference” means that the outer circumference of the breast-facing surface of the device is essentially capable of adhesion.

“Non-adhesive interior” means that remainder of the breast-facing surface of the device within the outer circumference is not capable of adhesion.

“Absorbent nursing pad” means a device capable of absorbing liquid, typically breast milk, used by human females during lactation. “Traditional absorbent nursing pad” refers to such a device, typically comprised of cotton or a natural or synthetic material having properties similar to cotton, and formed in an elliptical shape, ranging from approximately 2 inches in diameter.

“Thin” means substantially unnoticeable when worn under clothing.

“Translucent” means almost transparent; allowing light to pass through diffusely.

### SUMMARY OF THE INVENTION

The present invention is a device for holding in place an absorbent pad worn by lactating human females. The device is self contained in that it does not need the support of an ancillary garment such as a bra to hold it in place. The device comprises a thin, flexible and breathable, liquid impervious multi-layered shield that conforms to the shape of a human breast and is self adhesive. The device can be used in conjunction with a bra to assist in shaping the shield. However, the device is designed to adhere to the breast so that it does not require the use of ancillary clothing to perform it's function.

The shield comprises a plurality of silicon rubber compound layers laminated together. In general the layers are formed and cured independently of each other. There is no cross linking or merging of the separate layers. Generally, the innermost layer is formed and cured first. Subsequent layers are added on top of the innermost layer and then cured. In a preferred embodiment, the outermost layer is a silicon compound with a higher durometer than the inner layer. The outer layer provides rigidity and shape to the shield, and supports the layer or layers of low durometer compound. The silicon compounds are sufficiently stretchy in any direction so that a moderate pressure forces the present invention to conform to the shape of a human breast. Silicon has been chosen as the preferred material for an application of the invention that a user would have on the skin for extended periods of time. Silicon compounds have an inherent breathability or gas permeability which prevents irritation of the skin of the wearer. The present invention may also be made of other materials such as polymer gels. Although polymer gels do not have the gas permeability of silicon compounds, polymer gels would be suitable for applications such as use in fashion where the invention would not be worn for long periods of time.

The present invention has a low durometer silicon compound that forms the innermost layer next to the skin. Generally, a silicon compound known in the art as a silicon gel is used for the innermost surface. The inherent adhesive properties of a silicon gel provide a tacky inner circumference surface. The tackiness of the gel allows it to remain in position without the use of adhesives or having to be attached to an ancillary garment such as a bra.

In a preferred embodiment, the present invention is pre-formed to present an elliptical surface in cross section, enabling it to conform to the shape of a human breast. In other embodiments it is anticipated that the invention could pre-formed as to more closely match the shape of a particular breast. The adhesive properties of the silicon gel material allow the shield to adhere to the breast, on top of an absorbent pad. The shield material is sufficiently translucent as to be almost invisible underneath clothing. The thickness of the shield is such that virtually no additional bulk is added to the breast profile of the wearer.

In another embodiment, the present invention is a reusable, washable selectively adhesive cover for use by lactating women in combination with an absorbent nursing pad, said cover comprising a plurality of laminated silicone compound membranes, the outer laminate of a generally higher durameter than the inner laminate.

In yet another embodiment, the present invention is a reusable, washable selectively adhesive cover which is impermeable to liquid but permeable to gases.

In a further embodiment, the present invention is a reusable, washable selectively adhesive cover which is sufficiently adhesive that clothing is not required for use.

1. A reusable, washable selectively adhesive cover for use by lactating women in combination with an absorbent nursing pad, said cover comprising a plurality of laminated silicone compound membranes, an outer laminate of a generally higher durameter than an inner laminate.

2. The selectively adhesive cover of claim 1 that is impermeable to liquid but permeable to gases.

3. The selectively adhesive cover of claim 1 that is sufficiently adhesive that clothing is not required for use.

4. The selectively adhesive cover of claim 1 that is pre-formed to present an elliptical surface in cross section.
